(19) United States
(12) Patent Application Publication
(10) Pub. No.: US 2011/0251697 A1
(43) Pub. Date: Oct. 13, 2011

(54) FEMORAL HIP PROSTHESIS

(76) Inventors: Wui Chung, New South Wales (AU); Michael Neil, New South Wales (AU); Rami Sorial, New South Wales (AU); Craig Waller, New South Wales (AU); Michael Ribot, New South Wales (AU); Stephen Banks, New South Wales (AU)

(21) Appl. No.: 13/127,966

(22) PCT Filed: Sep. 4, 2009
(86) PCT No.: PCT/AU2009/001161

§ 371 Date: Jun. 27, 2011

(30) Foreign Application Priority Data
Nov. 5, 2008 (AU) 2008905702
Jun. 11, 2009 (AU) 2009902673

(51) Int. Cl. A61F 2/36

(52) U.S. Cl. 623/23.15

(57) ABSTRACT

A femoral hip prosthesis has a body having a body lateral face, a body medial face, a body front face and a body rear face. A stem extends longitudinally from a lower end of the body along a central stem axis. A neck extends from an upper end of the body along a central neck axis inclined at an obtuse angle to the central stem axis. The body lateral face is provided with a series of longitudinally spaced lateral face teeth transversely extending between the body front face and body rear face. Each of the lateral face teeth has an upper face inclined with respect to an adjacent tangent of the body lateral face by between 60 and 120 degrees.

### TECHNICAL FIELD

The present invention relates to the field of hip replacement prostheses, and particularly relates to a femoral hip prosthesis.

### BACKGROUND OF THE INVENTION

Various forms of femoral hip prostheses are known for use in hip replacement procedures, replacing diseased and/or damaged natural ball and socket hip joints. In such a procedure, the extreme upper end of the femur, which embodies the ball of the ball and socket hip joint, is removed. A femoral hip prosthesis is then implanted into the medullary canal of the femur with a neck of the prosthesis protruding therefrom for receipt of an artificial ball shaped head forming the male component of the artificial replacement hip joint. A cup shaped socket component is embedded into the hip bone so as to form the female component of the replacement hip joint.

The femoral hip prosthesis must endure various forces and bending moments, transferring loads between the leg and hip. In early designs, femoral hip prostheses relied on the use of cements to fix the prosthesis in place. Many designs, however, now utilise a cement free approach, relying on the use of a tapered stem which is press-fitted into the medullary canal, becoming wedged to effectively fix the femoral hip prosthesis in place. Femoral hip prostheses are also often provided with uneven surfaces which, over time, attract biological ingrowth of bony material to assist in permanent fixation of the prosthesis and to assist in load transfer between the prosthesis and native bone material.

Various present designs, however, do not adequately provide for effective load transfer between the hip prosthesis and native material under various typical load conditions.

### OBJECT OF THE INVENTION

It is the object of the present invention to substantially overcome or at least ameliorate at least one of the above disadvantages.

### SUMMARY OF THE INVENTION

In a first aspect, the present invention provides a femoral hip prosthesis comprising:

a body having a body lateral face, a body medial face, a body front face and a body rear face;

a stem extending longitudinally from a lower end of said body along a central stem axis;

a neck extending from an upper end of said body along a central neck axis inclined at an obtuse angle to said central stem axis;

wherein said body lateral face is provided with a series of longitudinally spaced lateral face teeth transversely extending between said body front face and said body rear face, each of said lateral face teeth having an upper face inclined with respect to an adjacent tangent of said body lateral face by between 60 and 120 degrees.

Typically, said upper face of each of said lateral face teeth is inclined substantially perpendicular to said central stem axis.

Preferably, said body medial face is provided with a series of longitudinally spaced medial face teeth transversely extending between said body front face and said body rear face, each of said medial face teeth having a lower face inclined with respect to an adjacent tangent of said body medial face by between 60 and 120 degrees.

Typically, said lower face of each of said medial face teeth is inclined substantially perpendicular to said tangent of said body medial face.

In a preferred form, said body front face and said body rear face each define a body major face, each said body major face being provided with a series of spaced primary major face teeth each extending from adjacent said body medial face, or from adjacent said lower end of said body, towards said body lateral face, each of said primary major face teeth having a lower face inclined with respect to an adjacent tangent of said body major face by between 60 and 120 degrees.

Typically, said lower face of each of said primary major face teeth is inclined substantially perpendicular to said tangent of said body major face.

Typically, each of said primary major face teeth extends upward across said body major face at an angle of between 30 and 60 degrees to said central stem axis.

More typically, each of said primary major face teeth extends upward across said body major face at an angle of about 45 degrees to said central stem axis.

Typically, each of said primary major face teeth that extends from adjacent said body medial face is substantially aligned with one of said medial face teeth.

In one embodiment, an upper lateral region of each said body major face is provided with a series of spaced secondary major face teeth extending from adjacent said body lateral face to adjacent a lateral end of one of said primary major face teeth, each of said secondary major face teeth having an upper face inclined with respect to an adjacent tangent of said body major face by between 60 and 120 degrees.

Typically, said upper face of each of said secondary major face teeth is inclined substantially perpendicular to said tangent of said body front face.

Typically, each of said secondary major face teeth extends across said front face substantially parallel to said primary major face teeth.

In a second aspect, the present invention provides a femoral hip prosthesis, comprising:

a body having a body lateral face, a body medial face, a body front face and a body rear face;

a stem extending longitudinally from a lower end of said body along a central stem axis;

a neck extending from an upper end of said body along a central neck axis inclined at an obtuse angle to said central stem axis;

wherein said medial face is provided with a series of longitudinally spaced medial face teeth transversely extending between said front face and said rear face, each of said medial face teeth having a lower face inclined substantially perpendicular to an adjacent tangent of said body medial face.

In a preferred form, said body front face and said body rear face each define a body major face, each said body major face being provided with a series of spaced primary major face teeth each extending from adjacent said body medial face, or from adjacent said lower end of said body, towards said body lateral face, each of said primary major face teeth having a lower face inclined with respect to an adjacent tangent of said body major face by between 60 and 120 degrees.

Typically, said lower face of each of said primary major face teeth is inclined substantially perpendicular to said tangent of said body major face.

Typically, each of said primary major face teeth extends upward across said body major face at an angle of between 30 and 60 degrees to said central stem axis.

More typically, each of said primary major face teeth extends upward across said body major face at an angle of about 45 degrees to said central stem axis.

Typically, each of said primary major face teeth that extends from adjacent said body medial face is substantially aligned with one of said medial face teeth.

In a third aspect, the present invention provides a femoral hip prosthesis, comprising:

a body having a body lateral face, a body medial face, a body front face and a body rear face, said body front face and said body rear face each defining a body major face;

a stem extending longitudinally from a lower end of said body along a central stem axis;

a neck extending from an upper end of said body along a central neck axis inclined at an obtuse angle to said central stem axis;

wherein each said body major face is provided with a series of spaced primary major face teeth extending from adjacent said body medial face or from adjacent said lower end of said body, towards said body lateral face, each of said primary major face teeth having a lower face inclined with respect to an adjacent tangent of said body major face by between 60 and 120 degrees;

further wherein each of said primary front face teeth extends upward across said body major face at an angle of between 30 and 60 degrees to said central stem axis.

Typically, each of said primary major face teeth extends upward across said body major face at an angle of about 45 degrees to said central stem axis.

Typically, said lower face of each of said primary major face teeth is inclined substantially perpendicular to said tangent of said body major face.

In one embodiment, an upper lateral region of each said body major face is provided with a series of spaced secondary major face teeth extending from adjacent said body lateral face to adjacent a lateral end of one of said primary major face teeth, each of said secondary major face teeth having an upper face inclined with respect to an adjacent tangent of said body major face by between 60 and 120 degrees.

Typically, said upper face of each of said secondary major face teeth is inclined substantially perpendicular to said tangent of said body major face.

Typically, each of said secondary major face teeth extends across said body major face substantially parallel to said primary major face teeth.

### BRIEF DESCRIPTION OF THE DRAWINGS

Preferred embodiments of the present invention will now be described, by way of example only, with reference to the accompanying drawings wherein:

FIG. 1 is an isometric view of a femoral hip prosthesis according to a first embodiment;

FIG. 2 is a front elevation view of the hip prosthesis of FIG. 1;

FIG. 3 is a left (lateral) side view of the hip prosthesis of FIG. 1;

FIG. 4 is a right (medial) side view of the hip prosthesis of FIG. 1;

FIG. 5 is a top plan view of the hip prosthesis of FIG. 1;

FIG. 6 is a bottom plan view of the hip prosthesis of FIG. 1;

FIG. 7 is an enlarged fragmentary front elevation view of the body and neck of the hip prosthesis of FIG. 1;

FIG. 8 is an enlarged front elevation view of the lateral region of the front face of the hip prosthesis of FIG. 1;

FIG. 9 is an enlarged front elevation view of the medial region of the front face of the hip prosthesis of FIG. 1;

FIG. 10 is a section front elevation view of a femoral hip prosthesis installation utilising the hip prosthesis of FIG. 1;

FIG. 11 is a front elevation view of a modified form of the hip prosthesis of FIG. 1;

FIG. 12 is an isometric view of a hip prosthesis according to a second embodiment;

FIG. 13 is a front elevation view of the hip prosthesis of FIG. 12;

FIG. 14 is a left (lateral) side view of the hip prosthesis of FIG. 12;

FIG. 15 is a right (medial) side view of the hip prosthesis of FIG. 12;

FIG. 16 is a top plan view of the hip prosthesis of FIG. 12; and

FIG. 17 is a bottom plan view of the hip prosthesis of FIG. 12.

### DETAILED DESCRIPTION OF THE PREFERRED EMBODIMENTS

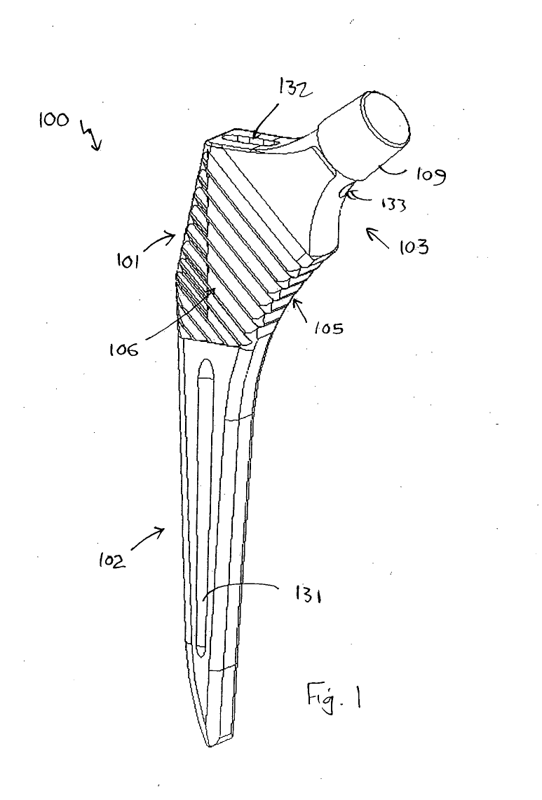

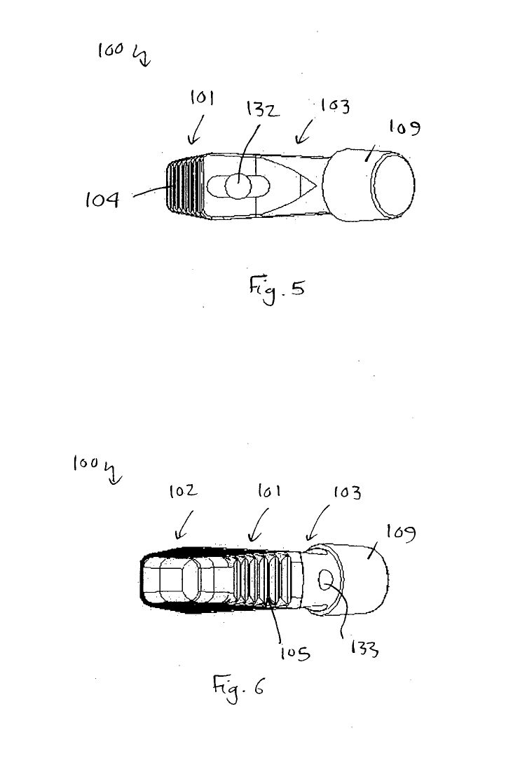

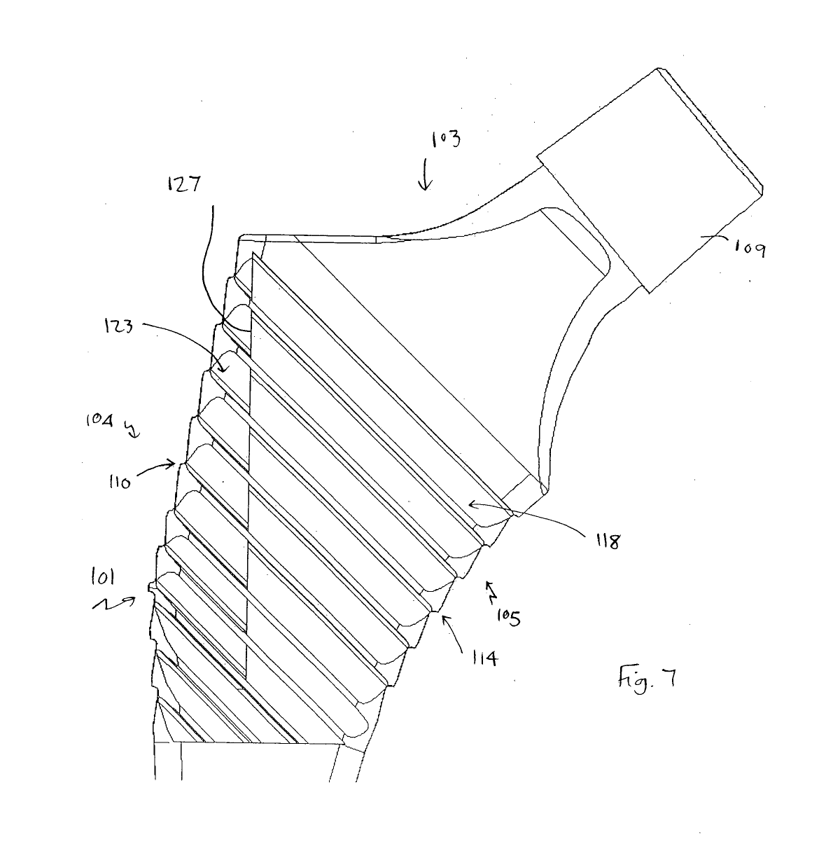

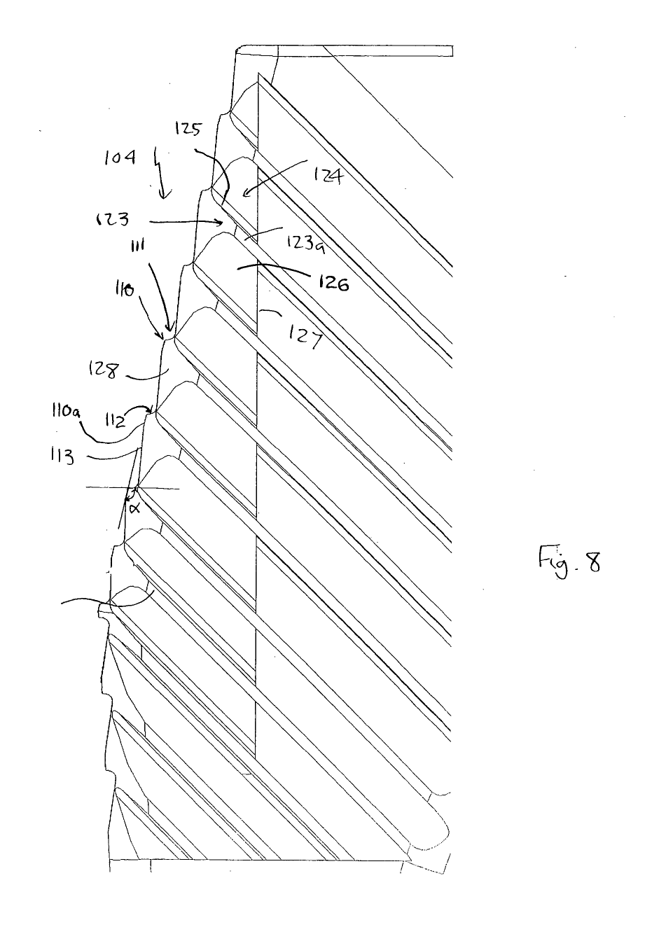

A femoral hip prosthesis 100 according to a first embodiment is depicted in FIGS. 1 to 9. The hip prosthesis 100 generally comprises a proximal body 101, a distal stem 102 extending longitudinally from the lower end of the body 101 along a central stem axis S and a neck 103 extending from an upper end of the body 101 along a central neck axis N. The central neck N is inclined at an obtuse angle (here typically about 130°) to the central stem axis S. The hip prosthesis 100 is here preferably formed from a titanium alloy. However, other biocompatible materials such as ceramics, composites and other metals and metal alloys having the required mechanical and material characteristics may be used.

The body 101 has a body lateral face 104 located on the lateral (left) side of the hip prosthesis 100 and a body medial (right) face 105 that is laterally separated from the body lateral face 104 and located on the medial (right) side of the hip prosthesis 100. The body 101 also has a body front face 106 located on the front (anterior) side of the hip prosthesis 100 and a body rear face 107 that is transversely separated from the body front face 106 and located on the rear (posterior) side of the hip prosthesis 100. The body front face 106 and body rear face 107 each define a body major face.

The distal (upper) end of the neck 103 is formed with a tapered end piece 109 for receipt of a ball shaped head in the usual manner. With the centre of the head being laterally offset from the central stem axis S (typically by 32 mm for a standard configuration and 38 mm for a high offset configuration prosthesis), the body 101 is subjected to bending loads, resulting from the typical vertical loads applied to the head. As a result, the present inventors have found that the medial region of the body 101 is typically subjected to compressive loads, whilst the lateral region (particularly the upper lateral region) of the body 101 is typically subjected to tensile loads. Whilst most known hip prostheses that are provided with uneven surface features to assist in load transfer between the hip prosthesis and femur are primarily directed at merely preventing the hip prosthesis from subsiding vertically deeper into the medullary canal, the femoral hip prosthesis 100 according to the first embodiment is provided with surface features in the form of various configurations of teeth to better provide for transfer of the typical loads acting on the hip prosthesis 100 to the femur.

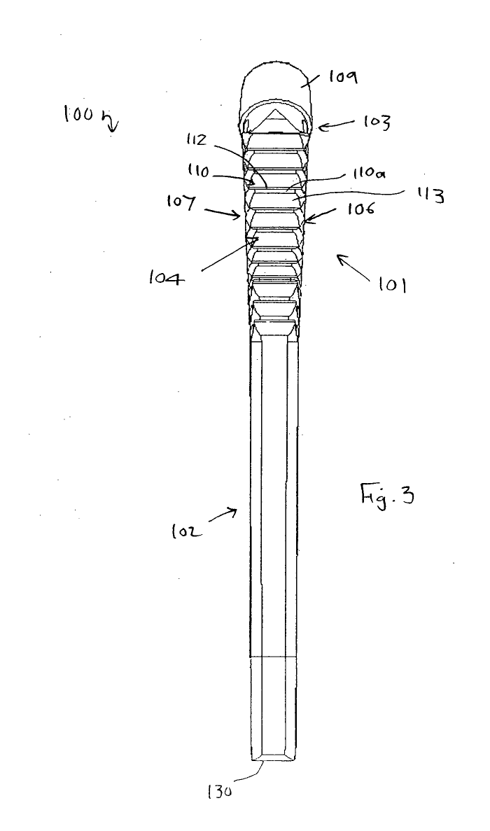

The bending moment acting on the hip prosthesis 100 may tend to rotate the hip prosthesis 100 within the medullary canal, tending to lift the lateral side of the hip prosthesis 100. Accordingly, to assist in transfer of the associated tension loads acting on the upper lateral region of the body 101, a series of longitudinally spaced lateral face teeth 110 are provided on the body lateral face 104, as best depicted in FIGS. 3, 7 and 8. The lateral face teeth 110 each transversely extend between the body front face 106 and body rear face 107 to provide a serrated surface on the body lateral face 104. The lateral face teeth 110 are formed (typically by machining) into the body lateral face 104, defining notches 111 therebetween for promoting ingrowth of native biological material of the femur. Each of the lateral face teeth 110 has an upper face 112 that is configured to transfer the tension load acting in the upper lateral region of the body 101 to the native material that grows into the notches 111 defined between the lateral face teeth 110.

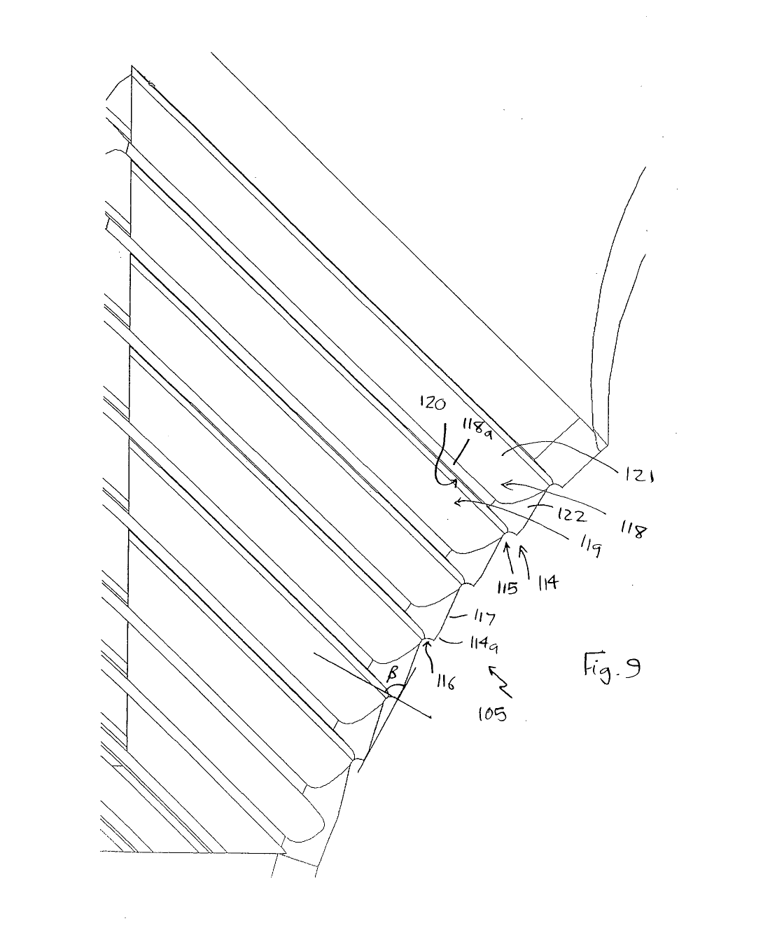

Referring specifically to FIG. 8, the upper face 112 of each of the lateral face teeth 110 is inclined with respect to an adjacent tangent of the body lateral face 104 by an angle α of between 60 degrees and 120 degrees. For manufacturability, the upper face 112 of each of the lateral face teeth 110 is typically inclined substantially perpendicular to the central stem axis S, although the most effective load transfer would generally be provided with the upper face 112 of each of the lateral face teeth 110 being inclined substantially perpendicular to the adjacent tangent of the lateral face 104. The lower face 113 of each of the lateral face teeth 110 is only offset from the tangent to the body lateral face 104 by a relatively small acute angle, and is typically oriented substantially parallel to the central stem axis S.

Between the upper face 112 and lower face 113 of each of the lateral face teeth 110 there is provided a relatively narrow flat outer surface 110a of the lateral tooth 110 that lies in the plane of the adjacent tangent of the body lateral face 104. This outer surface 110a allows for contact with the prepared femur during installation, and before the onset of bony ingrowth, over a finite area, rather than at a sharp point as might otherwise be the case if each of the lateral face teeth 110 was provided with a sharp juncture between the upper face 112 and lower face 113.

Each notch 111 typically has a depth of the order of 0.5 mm to 1.5 mm, and adjacent lateral face teeth 110 are typically spaced by about 3 mm to 6 mm.

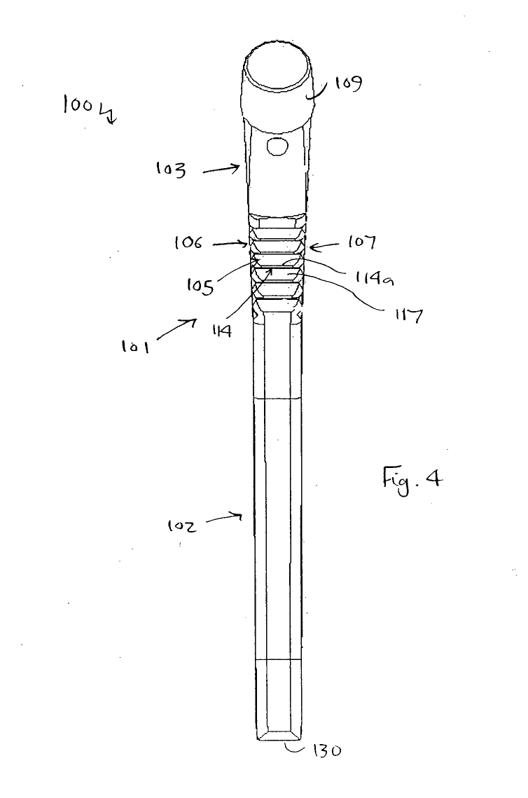

The loads acting on the medial region of the body 101 are typically compression loads that act to drive the hip prosthesis 101 deeper into the medullary canal. These forces are reacted by the calcar curve region of the femur, the curve of which is mimicked with the curve of the body medial face 105. To assist in transfer of the compression loads acting on the medial region of the body 101, a series of longitudinally spaced medial face teeth 114 are provide on the body medial face 105, as best depicted in FIGS. 4, 7 and 9. The medial face teeth 114 each transversely extend between the body front face 106 and body rear face 107 to provide a serrated surface on the body medial face 105. The medial face teeth 114 are again formed into the body medial face 105, defining notches 115 therebetween for promoting ingrowth of native biological material of the femur. Each of the medial face teeth 114 has a lower face 116 that is configured to transfer the compression load acting in the medial region of the body 101 to the native material that grows into the notches 115 defined between the medial face teeth 114.

Referring specifically to FIG. 9, the lower face 116 of each of the medial face teeth 114 is inclined with respect to an adjacent tangent of the medial face 105 by an angle β of between 60 degrees and 120 degrees. Particularly, here the lower face 116 of each of the medial face teeth 114 is inclined substantially perpendicular to the tangent to the medial face 105 so as to provide the most effective load transfer, given that the compression load acting on the body 101 adjacent to the medial face 105 will generally be in a direction substantially parallel to the medial face 105. The upper face 117 of each of the medial face teeth 114 is only offset from the tangent to the body medial face 105 by a relatively small acute angle, typically 5 to 10 degrees.

Between the upper face 117 and lower face 116 of each of the medial face teeth 114 there is provided a relatively narrow flat outer surface 114a of the medial tooth 114 that lies in the plane of the adjacent tangent of the body medial face 105.

Each notch 115 typically has a depth of the order of 0.5 mm to 1.5 mm, and adjacent medial face teeth 114 are typically separated by about 3 mm to 6 mm.

The present inventors have found that, under typical loading conditions, the compression loads acting on the medial region of the body extend partway across the body front and body rear faces 106, 107. The lower lateral region of the body 101 may also be subject to compression loads, whilst the upper lateral region of the body 101 typically carries tension loads.

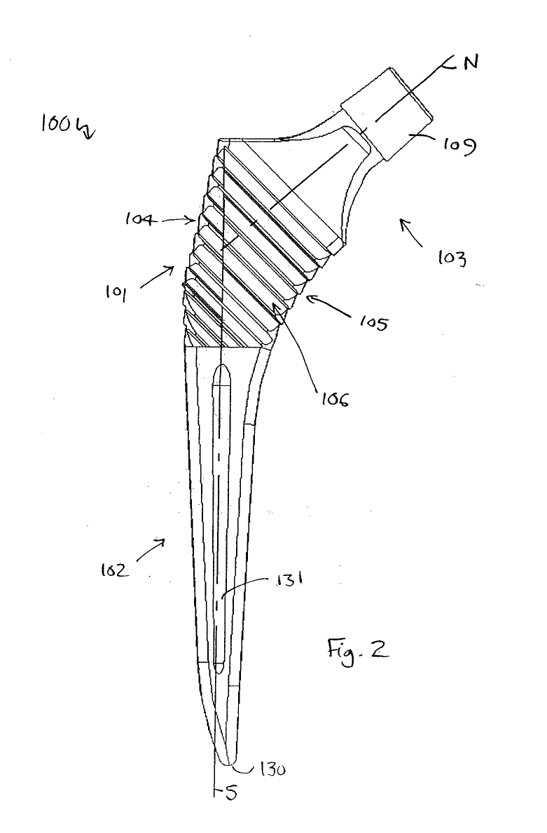

To assist in transfer of the compression loads acting in the medial and lower lateral regions of the body 101, a series of spaced primary major face teeth 118 are provided on the body major faces (that is, the body front face 106 and body rear face 107), as best depicted in FIGS. 2, 7 and 9. In the arrangement depicted, the seven uppermost primary major face teeth 118 extend from adjacent the body medial face 105 towards the body lateral face 104. The next two primary major face teeth 118 each extend from adjacent the lower end of the body 101 towards the body lateral face 104, whilst the two lowermost primary major face teeth 118 extend from adjacent the lower end of the body 101 to adjacent the body lateral face 104. The primary major face teeth 118 are each formed into the body major face 106, 107, defining notches 119 therebetween for promoting growth of native biological material of the femur. Each of the primary major face teeth 118 is configured similar to the medial face teeth 114, each having a lower face 120 that is configured to transfer the compression load acting locally on the body 101 to the native material that grows into the notches 119 defined between the primary major face teeth 118.

The lower face 120 of each of the primary major face teeth 118 is inclined with respect to an adjacent tangent of the major face 106, 107 by between 60 degrees and 120 degrees so as to provide for effective load transfer. More particularly, the lower face 120 of each of the primary major face teeth 118 is inclined substantially perpendicular to the tangent to the body major face 106, 107. The upper face 121 of each of the primary major face teeth 117 is only offset from the tangent to the body major face 106, 107 by a relatively small acute angle.

Between the upper face 121 and lower face 120 of each of the primary major face teeth 117 there is provided a relatively narrow flat outer surface 117a of the primary major face tooth 117 that lies in the plane of the adjacent tangent of the body major face 106, 107.

Each notch 119 typically has a depth of the order of 0.5 mm to 1.5 mm. The primary major face teeth 118 that extend from adjacent the body medial face 105 are each typically aligned with one of the medial face teeth 114. So as to avoid a sharp point at the junction between each of the medial face teeth 114 and the aligned primary major face tooth 118, the junction 122 between each medial tooth 114 and primary major face tooth 117 is rounded or chamfered, thereby reducing the possibility of the body 101 catching during the implantation process.

The improved load transfer between the medial face 105 and the femoral calcar resulting from the medial teeth 114 will increase the compression load acting on the femoral calcar and, accordingly, growth of the bond stock in this area will be encouraged.

Each of the primary major face teeth 118 extends across the body major face 106, 107 upwards towards the body lateral face 104 at an angle of between 30 degrees and 60 degrees to the central stem axis S, so as to better position the primary major face teeth 118 for transferring the compression loads acting on the body 101, which extend roughly diagonally from the neck 109 through the body 101. It is particularly preferred that each of the primary major face teeth 118 extends upward across the major face 106, 107 at an angle of about 45 degrees to the central stem axis S.

To assist in transfer of the tension loads that act on the upper lateral region of the body 101, a series of spaced secondary major face teeth 123 are provided on each of the body major faces 106, 107, as best depicted in FIGS. 2, 7 and 8. The secondary major face teeth 123 each extend from adjacent the lateral face 104 to adjacent the lateral end of one of the primary major face teeth 118. The transition line 127 defined by the junctions between the primary major face teeth 118 and secondary major face teeth 112 roughly correlates to a boundary between regions of the body 101 which are subject to compressive loading (to the lateral and lower side of the transition line 127) and tension loading (to the upper lateral side of the transition line 127). There the transition line 127 is approximately aligned with the stem central axis S.

Each of the secondary major face teeth 123 is formed into the body major faces 106, 107 defining notches 124 therebetween for promoting ingrowth of native biological material of the femur. Each of the secondary major face teeth 123 is configured similarly to each of the lateral face teeth 110, having an upper face 125 that is configured to transfer tension loads acting in the upper lateral region of the body 101 to the native material that grows into the notches 124 defined between the secondary major face teeth 123.

The upper face 125 of each of the secondary major face teeth 123 is inclined with respect to an adjacent tangent of the body major face 106, 107 by between 60 degrees and 120 degrees, and most typically is inclined substantially perpendicular to the tangent of the body major face 106, 107. The lower face 126 of each of the secondary major face teeth 122 is only offset from the tangent to the body major face 106, 107 by a relatively small acute angle.

Between the upper face 125 and lower face 126 of each of the secondary major face teeth 123 there is provided a relatively narrow flat outer surface 123a of the secondary major face tooth 123 that lies in the plane of the adjacent tangent of the body major face 106, 107.

Each notch 124 typically has a depth of the order of 0.5 mm to 1.5 mm. Each of the secondary major face teeth 123 is typically substantially aligned with one of the lateral face teeth 110. The junction 128 between each of the secondary major face teeth 123 and lateral face teeth 110 is again typically rounded or chamfered so as to avoid a sharp point being defined at the junction.

Whilst the notches 119 and 124 defined by the primary major face teeth 118 and secondary major face teeth 123 respectively each have a constant depth of the order of 0.5 mm to 1.5 mm extending across each body major face 106, 107, it is envisaged that the depth of the notches 119, 124 may decrease toward the transition line 127.

Whilst, on the hip prosthesis 101 of the first embodiment, both primary and secondary major face teeth 118, 123 are employed, it is envisaged that one or other of these forms of teeth may be omitted, with the remaining configuration of teeth potentially extending across the entire major face, depending upon the specific configuration of the hip prosthesis and anticipated loading.

When viewing the prosthesis 101 from either the lateral side (see FIG. 3) or medial side (see FIG. 4), it can be seen that the hip prosthesis 101 is symmetric about a central plane. The stem tapers towards the distal (lower) tip 130 typically with an included angle of about 0 to 5 degrees whilst the body has a greater taper, typically with an included angle of between 4 to 10 degrees. As a result, load transfer between the hip prosthesis 101 and surrounding femur is primarily concentrated in the body. The body 101 and stem 102 are also tapered when viewed from the front or rear (see FIG. 2), with the body medial face 105 generally following the curve of the femoral calcar, as discussed above. The stem 102 typically has its distal tip 130 offset towards the medial side so as to reduce the possibility of the distal tip 130 impinging on the endosteum if the bending moments acting on the hip prosthesis 101 result in some minor rotation of the hip prosthesis 101 within the medullary canal. Such impingement on the endosteum may otherwise result in thigh pain. The stem 102 may also be provided with one or more longitudinally extending grooves 131 in the major faces 106, 107 so as to reduce the stiffness of the stem 102 and better mimic the stiffness of the native femur.

The neck 103 of the hip prosthesis 101 is provided with a cavity 132 in the upper end thereof for receipt of insertion tools utilised during hip replacement surgery to locate the hip prosthesis 101. The cavity 132 may be threaded for receipt of a threaded insertion tool. The neck 103 may also be provided with a dimple 133 on its underside to assist in location of a stem removal instrument that engages the neck 103 adjacent the end piece 109 to remove the hip prosthesis 101 if required. The neck 103 may also be tapered adjacent the end piece 109 so as to reduce the possibility of the cup component impinging on the neck 103 during its range of movement in use.

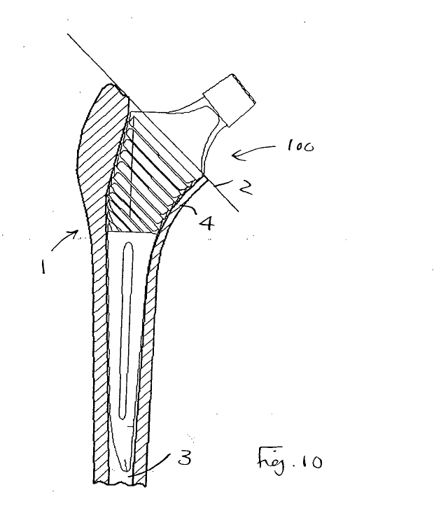

FIG. 10 depicts the hip prosthesis 100 in situ within a prepared femur 1. The hip prosthesis 101 is installed in the usual manner for a cementless prosthesis design. Firstly, x-ray templating of the native femur 1 is conducted prior to commencement of surgery, so as to allow for selection of the appropriate hip prosthesis sizing and configuration. A femoral neck osteotomy 2 is then conducted, with the upper extremity of the femur being cut, typically at an angle of 45 degrees from the centre line of the femur. The medullary canal 3 is then prepared for the hip prosthesis installation. Femoral broaching is then conducted, filing and compressing the femoral bone within the medullary canal 3 using a broach in accordance with the usual procedure. The calcar 4 is then prepared, following which a trial reduction is conducted utilising a trial hip prosthesis and trial head, which are all assembled into place against an acetabular cup to trial the replacement hip joint. The hip prosthesis 101 is then inserted into the medullary canal into its final position, and a final trial reduction is conducted utilising a trial ball against an acetabular cup. Once the surgeon is satisfied with the implantation of the stem and the stem geometry, the final head is assembled onto the end piece 109 of the neck 103 of the hip prosthesis 101, and the final reduction is completed.

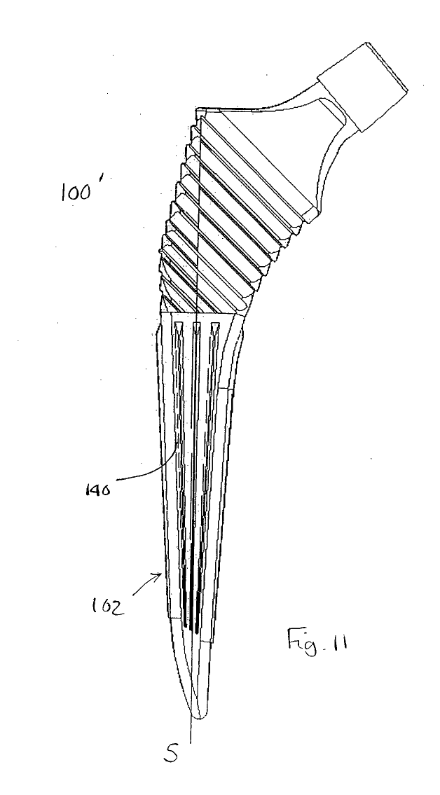

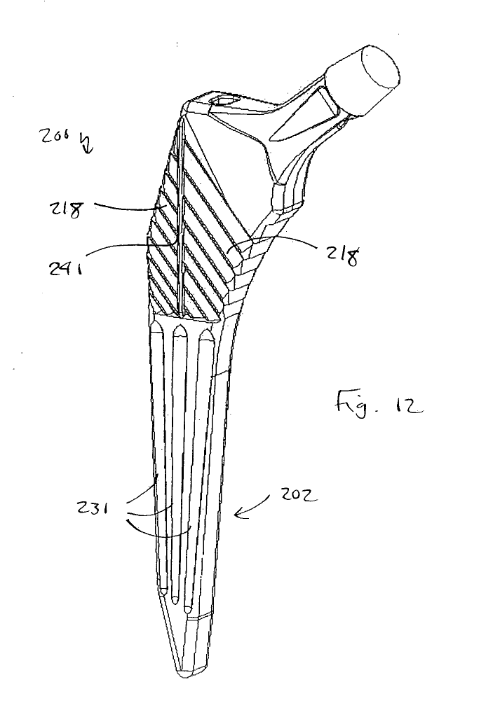

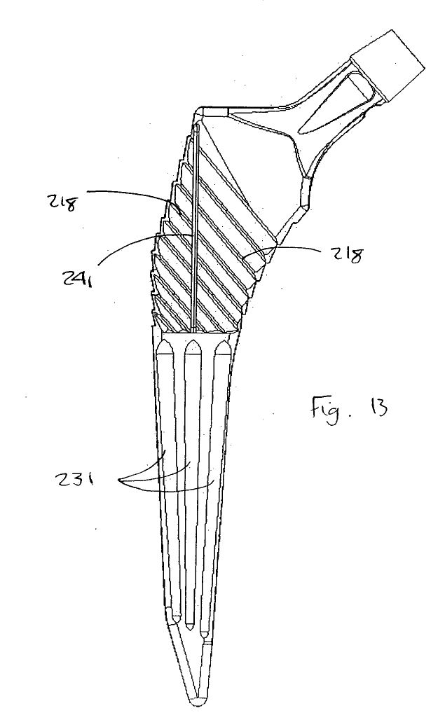

FIG. 11 depicts a modified form of the femoral hip prosthesis 100′ of the first embodiment. The hip prosthesis 100′ utilises a series of longitudinally extending ribs 140 extending along each face of the stem 102. These ribs 140 provide torsional stability to the stem 102, inhibiting angular displacement of the hip prosthesis 100′ about the central stem axis S.

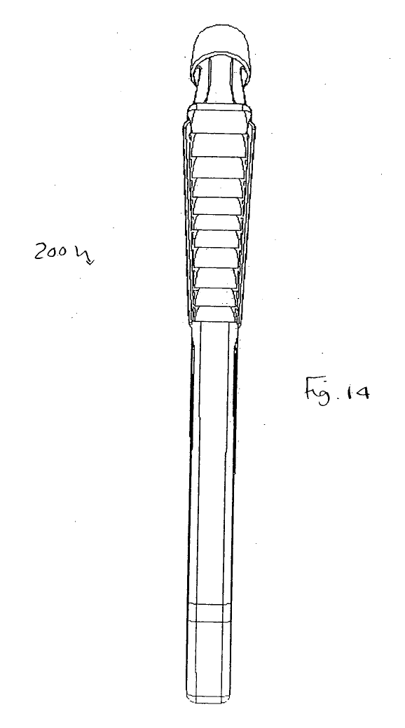

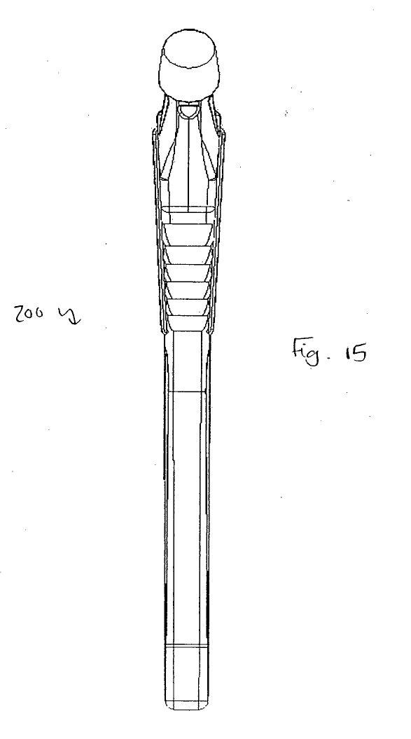

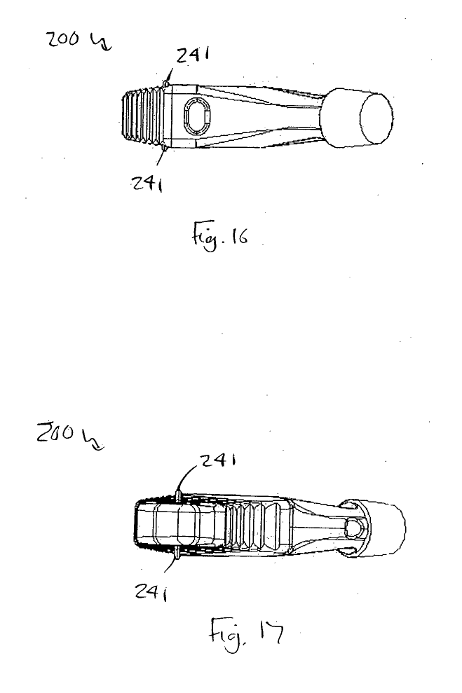

A femoral hip prosthesis 200 according to a second embodiment is depicted in FIGS. 14 through 17. The hip prosthesis 200 of the second embodiment is substantially similar to the femoral hip prosthesis 100 of the first embodiment described above, and like or equivalent features are provided with the same reference numerals that are utilised in the above description of the hip prosthesis 100 of the first embodiment, increased by 100.

In the hip prosthesis 200, the primary major face teeth 218 extend from either adjacent the medial face 205 or adjacent the lower end of the body 201 right across each body major face 206, 207 to adjacent the body lateral face 204. As a result, the secondary major face teeth are omitted. The primary major face teeth 218 are interrupted by a longitudinally extending rib 241 located on each of the major faces 206, 207. The ribs 241 assist in torsional control, inhibiting torsional displacement of the hip prosthesis 101 about the central stem axis S.

The femoral hip prosthesis 200 can also be seen to be provided with a series of three longitudinally extending grooves 231 on each of the front and rear faces of the stem 202. The hip prosthesis 200 of the second embodiment is otherwise substantially identical to the hp prosthesis 100 of the first embodiment. A person skilled in the art will appreciate that various other modifications of the hip prosthesis may be made to suit specific applications.

1. A femoral hip prosthesis comprising:
a body having a body lateral face, a body medial face, a body front face and a body rear face;
a stem extending longitudinally from a lower end of said body along a central stem axis; and
a neck extending from an upper end of said body along a central neck axis inclined at an obtuse angle to said central stem axis;
wherein said body lateral face is provided with a series of longitudinally spaced lateral face teeth transversely extending between said body front face and said body rear face substantially perpendicular to said central stem axis, each of said lateral face teeth having an upper face inclined with respect to an adjacent tangent of said body lateral face by between 60 and 120 degrees.

2. The prosthesis of claim 1, wherein said upper face of each of said lateral face teeth is inclined substantially perpendicular to said central stem axis.

3. The prosthesis of claim 1, wherein said body medial face is provided with a series of longitudinally spaced medial face teeth transversely extending between said body front face and said body rear face, each of said medial face teeth having a lower face inclined with respect to an adjacent tangent of said body medial face by between 60 and 120 degrees.

4. The prosthesis of claim 3, wherein said lower face of each of said medial face teeth is inclined substantially perpendicular to said tangent of said body medial face.

5. The prosthesis of claim 3, wherein said body front face and said body rear face each define a body major face, each said body major face being provided with a series of spaced primary major face teeth each extending from adjacent said body medial face, or from adjacent said lower end of said body, towards said body lateral face, each of said primary major face teeth having a lower face inclined with respect to an adjacent tangent of said body major face by between 60 and 120 degrees.

6. The prosthesis of claim 5, wherein said lower face of each of said primary major face teeth is inclined substantially perpendicular to said tangent of said body major face.

7. The prosthesis of claim 5, wherein each of said primary major face teeth extends upward across said body major face at an angle of between 30 and 60 degrees to said central stem axis.

8. The prosthesis of claim 5, wherein each of said primary major face teeth that extends from adjacent said body medial face is substantially aligned with one of said medial face teeth.

9. The prosthesis of claim 5, wherein an upper lateral region of each said body major face is provided with a series of spaced secondary major face teeth extending from adjacent said body lateral face to adjacent a lateral end of one of said primary major face teeth, each of said secondary major face teeth having an upper face inclined with respect to an adjacent tangent of said body major face by between 60 and 120 degrees.

10. The prosthesis of claim 9, wherein said upper face of each of said secondary major face teeth is inclined substantially perpendicular to said tangent of said body front face.

11. The prosthesis of claim 9, wherein each of said secondary major face teeth extends across said front face substantially parallel to said primary major face teeth.

12. A femoral hip prosthesis, comprising:
a body having a body lateral face, a body medial face, a body front face and a body rear face, said body front face and said body rear face each defining a body major face;
a stem extending longitudinally from a lower end of said body along a central stem axis; and
a neck extending from an upper end of said body along a central neck axis inclined at an obtuse angle to said central stem axis;
wherein each said body major face is provided with a series of spaced primary major face teeth extending from adjacent said body medial face or from adjacent said lower end of said body, towards said body lateral face, each of said primary major face teeth having a lower face inclined with respect to an adjacent tangent of said body major face by between 60 and 120 degrees;
further wherein each of said primary front face teeth extends upward across said body major face at an angle of between 30 and 60 degrees to said central stem axis; and
further wherein an upper lateral region of each said body major face is provided with a series of spaced secondary major face teeth extending from adjacent said body lateral face to adjacent a lateral end of one of said primary major face teeth, each of said secondary major face teeth having an upper face inclined with respect to an adjacent tangent of said body major face by between 60 and 120 degrees.

13. The prosthesis of claim 12, wherein each of said primary major face teeth extends upward across said body major face at an angle of about 45 degrees to said central stem axis.

14. The prosthesis of claim 12, wherein said lower face of each of said primary major face teeth is inclined substantially perpendicular to said tangent of said body major face.

15. The prosthesis of claim 12, wherein said upper face of each of said secondary major face teeth is inclined substantially perpendicular to said tangent of said body major face.

16. The prosthesis of claim 12, wherein each of said secondary major face teeth extends across said body major face substantially parallel to said primary major face teeth.

17. The prosthesis of claim 12, wherein said medial face is provided with a series of longitudinally spaced medial face teeth transversely extending between said front face and said rear face, each of said medial face teeth having a lower face inclined substantially perpendicular to an adjacent tangent of said body medial face.

18. The prosthesis of claim 17, wherein each of said primary major face teeth that extends from adjacent said body medial face is substantially aligned with one of said medial face teeth.

19.-22. (canceled)
